(19) United States
(12) Patent Application Publication
(10) Pub. No.: US 2006/0052942 A1
(43) Pub. Date: Mar. 9, 2006

(54) Integrated knowledge-based reverse engineering of metabolic pathways

(76) Inventors: Shuo-Huan Hsu, W. Lafayette, IN (US); Priyan R. Patkar, (US); Santhoji R. Katare, Inkster, MI (US); John A. Morgan, West Lafayette, IN (US); Venkat Venkatasubramanian, W. Lafayette, IN (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/134,907

(22) Filed: May 23, 2005

Related U.S. Application Data
(60) Provisional application No. 60/573,647, filed on May 21, 2004.

(51) Int. Cl. C12P 7/06; G06F 19/00; C12N 1/21

(52) U.S. Cl. 702/19; 435/161; 435/252.33

(57) ABSTRACT

A method of enhancing the yield of a product is provided. A host organism that is adapted to produce the product as a function of metabolism in the presence of a substrate is selected and a plurality of optimal reaction pathways are determined by searching a database and executing an optimization algorithm. The optimal reaction pathways take place in the host organism to produce the product from the substrate and are ranked and enumerated. The optimization algorithm comprises a flux balance analysis and maximizes the yield of the product.

### FIELD OF THE INVENTION

The present invention relates generally to metabolic reaction pathways and more particularly to identifying optimal pathways from a large database of reactions.

### BACKGROUND

Traditional scientific model building processes are often slow and inefficient in today's era of bioinformatics and systems biology. Such inefficiencies can be seen, for instance, in processes for postulating a hypothesis to explain data, as these processes require the hypothesis to be translated into a model, validated against limited data, and manually refined for model-data mismatch. Accordingly, the pace of data generation in today's society has warranted the need for automated tools that can aid a human expert to rapidly and efficiently complete model building tasks.

Rational and automated methodologies (as opposed to traditional guess-and-test procedures) for systematically understanding the dynamics of a cell are particularly important, as these structures require the ability to reverse engineer metabolic reaction networks, as well as construct and analyze transcriptome, proteome and metabolic data surrounding the interactions of the cellular species. However, large specie numbers, as well as environmental interactions and variability make this task particularly difficult.

Attempts to automate the process for constructing metabolic reaction pathways have received considerable attention within the scientific community. Knowledge of steady state conditions has fostered these attempts, particularly as obtaining dynamic metabolic cellular data has been experimentally difficult. For instance, attempts to synthesize metabolic pathways by using artificial intelligence (AI) processes were first addressed in 1988 by Seressiotis and Bailey (see Seressiotis, A. and Bailey, J. E. (1988) Biotechnology and Bioengineering, 31, 587-602). Given databases of enzyme and substrate description, AI search algorithms were designed to identify qualitative feasible pathways (see Mavrovouniotis, M. L., Stephanopoulos, G. and Stephanopoulos, G. (1990) Biotechnology and Bioengineering, 36, 1119-1132). Methodologies for synthesizing and analyzing metabolic pathways according to this approach; however, are not guaranteed for optimality or completeness. In addition to AI processes, graph theoretical approaches have also been applied to construct metabolic or reaction networks by using stoichiometric information (see Arita, M. (2000) Simulation Practice and Theory, 8, 109-125; Seo, H., Lee, D.Y., Park, S., Fan, L. T., Shafie, S., Bertok, B. and Friedler, F. (2001) Biotechnology letters, 23, 1551-1557; and Fan, L. T., Bertok, B. and Friedler, F. (2002) Computers and Chemistry, 26, 265-292). While this approach may be used to enumerate all feasible pathways, the algorithms are only efficient for relatively small networks.

Another valuable method of steady state analysis of metabolic reaction pathways is the Flux Balance Analysis, “FBA,” which formulates the analysis as a linear program (see Varma, A. and Palsson, B. O. (1993) Journal of Theoretical Biology, 165, 477-502). There are several applications of FBA, such as finding minimal reaction sets under different environments (see Burgard, A. P., Vaidyaraman, S. and Maranas, C. D. (2001) Biotechnology Progress, 17, 791-797), estimating the performance subject to gene addition or deletions (see Burgard, A. P. and Maranas, C. D. (2001) Biotechnology and Bioengineering, 74, 364-375), and testing hypothesized metabolic objective functions (see Burgard, A. P. and Maranas, C. D. (2003) Biotechnology and Bioengineering, 82, 670-677). It is also possible to have multiple solutions for the same objective value, such as by utilizing an algorithm to enumerate all possible linear programming solutions (see Lee, S., Phalakornkule, C., Domach, M. M. and Grossmann, I. E. (2000) Computers and Chemical Engineering, 24, 711-716). However, only the upper/lower bound of the fluxes can be expected from this analysis. Thus, it would be desirable to overcome these and other shortcomings of the prior art.

### SUMMARY OF THE INVENTION

The present invention provides a framework for engineering a metabolic reaction pathway to optimize production of a desired product. The method involves engineering a host organism in which the product is produced to maximize the yield of the product while at the same time optimizing parameters such as ATP production and cost of engineering the host organism.

In one form thereof, the present invention provides a method of enhancing product. The inventive method includes the step of selecting a host organism adapted to produce the product as a function of metabolism in the presence of a substrate. A plurality of optimal reaction pathways is determined by searching a database and executing an optimization algorithm. The optimal reaction pathways take place in the host organism to produce the product from the substrate. The optimum reaction pathways are then ranked and enumerated.

According to another form thereof, the present invention provides a method of ranking a plurality of metabolic reactions. The inventive method includes the step of accessing a database containing a plurality of metabolic reactions. A flux balance analysis is conducted by solving a mixed-integer program calculation to determine the plurality of optimal reaction pathways from the plurality of metabolic reactions. The optimal reaction pathways are then ranked and enumerated by one or more of, evaluating kinetics, evaluating ATP production, and minimizing changes to the host organism.

In exemplary forms, the present invention contemplates not only selecting a host organism, but also genetically modifying or engineering it to create a “hybrid” host organism. For example, the host organism may include reaction pathways that are undesirable because they slow the reaction process or produce poor yields. In specific forms, the present invention may modify genes of the host organism by adding or subtracting an enzyme.

The selection of the host organism, substrate, and optimization of the host can all be provided as outputs by a computer programmed to perform the method of the present invention, in which event the method is entirely automated. Alternatively, certain process variables, such as the particular host organism to be used, can be manually provided as inputs.

According to specific illustrations, the step of determining the plurality of optimum pathways is automated and is based upon a flux balance analysis (“FBA”). More specifically, the objective of the FBA is maximizing the flux of the product to be produced. A novel mixed-integer program described in more detail below is used to enumerate the multiple optimal pathways.

In other specific embodiments, the ranking of the enumerated optimal pathways involves using different criteria to discriminate between them. For instance, a pathway producing more ATP is favorable over other optima because it is energetically more productive. Another important consideration can be the difficulty or effort required to genetically engineer a candidate pathway topology. If the topology is easy to engineer, the engineered strain can be made relatively fast, which means this pathway is practically obtainable. The number of genes to knockout or add can be used as a measure of the genetic engineering “cost” or effort associated with a pathway.

### BRIEF DESCRIPTION OF DRAWINGS

The above-mentioned aspects of the present invention and the manner of obtaining them will become more apparent and the invention itself will be better understood by reference to the following description of the embodiments of the invention taken in conjunction with the accompanying drawings, wherein:

FIG. 1 is a graphical display of a network of exemplary metabolic pathways available for producing a product in accordance with the present invention;

FIG. 2 is a graphical display illustrating a method incorporating the present invention;

FIG. 3 is a flow diagram illustrating an exemplary process for enumerating multiple topologies with the same theoretical yield in accordance with the present invention;

FIG. 4 depicts a plurality of integer value formulations for selecting an optimal metabolic reaction pathway with a Flux Balance Analysis in accordance with the present invention

FIG. 5 is a chart illustrating ATP production distribution for different topologies according to the present invention;

FIGS. 6(a) and 6(b) depict two exemplary topologies for producing ethanol according to the glycolysis pathways of EMP (Embden-Meyerhof-Parnas) and ED (Entner-Doudoroff), respectively;

FIG. 6(c) is an exemplary pathway including the TCA cycle and used to generate ATP to satisfy the maintenance constraint according to the present invention; and

FIGS. 7(a) and 7(b) depict exemplary pathways for producing succinate from E. coli in the presence of a glucose substrate in accordance with the present invention.

Corresponding reference characters indicate corresponding parts throughout the several views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

Embodiments incorporating the present invention generally relate to knowledge-based reverse engineering methods that are used to engineer metabolic pathways for value added biotransformations. The metabolic pathways are constructed using steady state analyses, such as the flux balance analysis approach, to produce theoretical yields (i.e., products) in an efficient and commercializable manner. Known reactions, organisms, processes, etc. are analyzed according to this approach and new and more efficient processes for producing a commodity item are generated, thereby reducing the associated costs for producing such items. These processes can modify an organism (for example, by genetic modification), alter the type of organism used and/or alter the steps in the process or the biomaterials used, etc. to make the overall outcome more productive and cost efficient. In certain exemplary embodiments, multiple solutions are used and enumerated, and rules for screening candidate pathways are applied to reduce the number of candidates. Moreover, computational flux balances of central metabolic host organisms are used to show the addition of enzymes to a metabolic pathway and increase the theoretical yield of the product from a specified substrate.

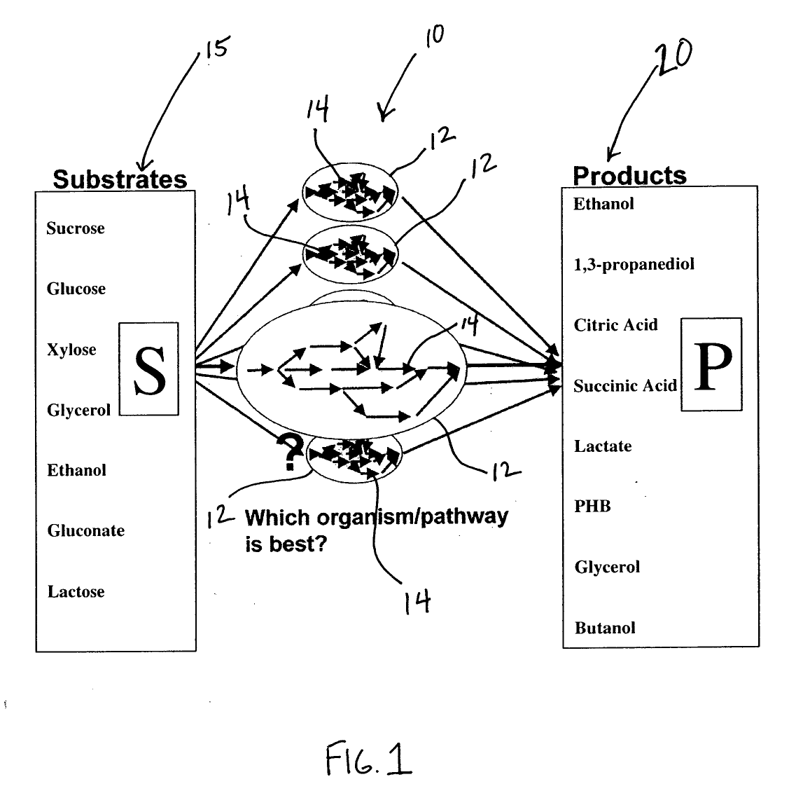

In exemplary embodiments according to the present invention, the yield of a product is enhanced from a substrate by optimizing the flux of a product by enumerating and ranking multiple reaction pathways. According to this embodiment, a host organism in which the substrate reacts to form the product as a function of metabolism is selected, and a plurality of reaction pathways associated with the host are enumerated and ranked for optimization of the product yield (i.e., the framework will identify all optimal reaction pathways, list them and rank them according to the criteria explained elsewhere). For example, referring now to FIG. 1, numerous metabolic reaction pathways 10 associated with host organisms 12 containing genetic information 14 (e.g., genes) are available to form a product 20 from a substrate 15 according to the present invention. Exemplary substrates according to the present invention include sucrose, glucose, xylose, glycerol, ethanol, gluconate and lactose, while exemplary products include ethanol, 1,3-propanediol, citric acid, succinic acid, lactate, PHB, glycerol and butanol. The metabolic reaction pathways are available in metabolic network databases (e.g., EMP, MetaCyc, UM-BBD, KEGG, BRENDA, BioCyc, etc.) and can be accessed, for instance, by a computer aided pathway search. As is known within the art, these network databases contain comprehensive collections of information on all known metabolic reactions and are publicly accessible for analysis and review.

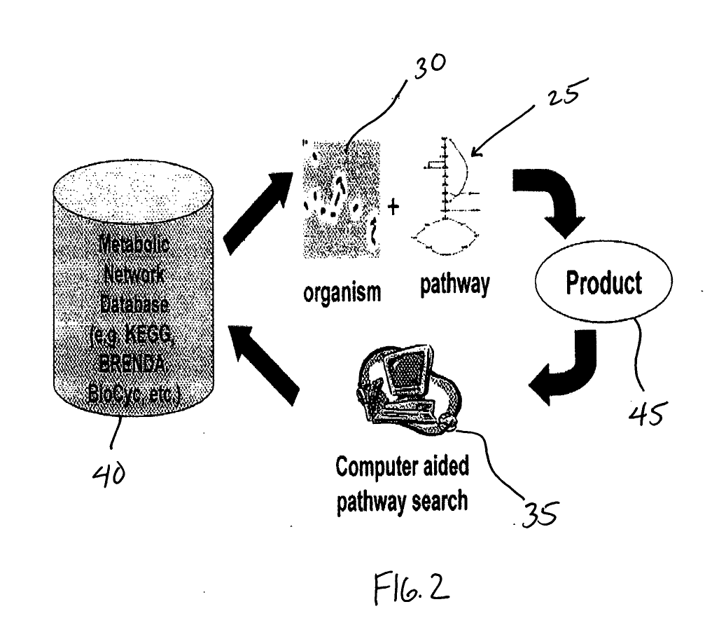

Referring now to FIG. 2, the metabolic reaction pathway of a host organism is optimized to increase the theoretical yield of a product. According to this exemplary illustration, metabolic network database 40 is accessed via computer aided pathway search 35 to identify and enumerate all metabolic reaction pathways available for producing product 45. More particularly, host organism 30 serves as a support medium in which product 45 is produced. In an exemplary illustration, while host organism 30 produces product 45 as part of its natural function when in the presence of a substrate (not shown), its metabolic reaction pathway 25 may not be the optimal route for producing the product (i.e., it is kinetically inefficient and/or has high engineering costs to the organism). As such, a computer aided pathway search 35 is performed on the metabolic network database 40 to identify and enumerate all available reaction pathways for producing the product. Once these reaction pathways have been identified, the pathways are ranked and the optimum pathway determined. The ranking of the enumerated optimal pathways is determined by discriminating among them by using different criteria, such as ATP production potential, kinetic evaluations, engineering costs and/or evaluating changes to the host organism. With this information, a metabolic engineer can then modify the host organism's metabolic reaction pathway 25 by inserting genetic information, such as genes or enzymes, from a second host source into the pathway framework and thereby allow the host organism to produce greater product yield. The enumeration and ranking processes according to this exemplary illustration are determined by utilizing integer value formulations, which will be explained in greater detail below. In specific embodiments, the integer value formulation is utilized to effectively rank the optimal enumerated pathways by considering factors such as the ATP production potential and engineering costs of the pathways.

In exemplary embodiments, the above process can be achieved with the FBA approach. FBA processes require information about the stoichiometric ratio reactions, requirements for growth, and the measurement of a few strain-specific parameters, and are based on the fact that metabolic transients are typically rapid as compared to cellular growth rates and environmental changes. Therefore, a pseudo-steady state assumption can be applied to lead to the following flux balance equation:
S·v=0 (1)
where S is the matrix containing the stoichiometric ratios of the metabolic reactions and v is the flux vector. In general, this equation is underdetermined since the number of fluxes normally exceeds the number of metabolites. The problem can be solved as a linear program to obtain a rational solution by specifying an objective, such as maximizing the organism growth, maximizing the yield of a metabolite, etc.

The objective of a typical flux balance analysis focuses on the maximized flux of biomass production within an organism, which is perceived to be an evolutionary objective. However, there are other rational objective choices of a flux balance analysis, such as maximizing the flux of a certain metabolite or ATP. According to this approach, the quantity of interest is the theoretical yield of some product P and the objective function is set to be the maximization of the flux vₚ, constrained by the flux vₛ of a certain substrate S. The theoretical yield is represented by |vₚ/vₛ| and the substrate flux vₛ is equal to −1 (the negative sign indicating the consumption of the metabolite). The complete formulation is as follows:
max vₚ
subject to

$$\begin{matrix}\begin{matrix}{\sum }_{j} {s}_{ij} {v}_{j} = 0 & & ∀ i ∈ {M}_{i}\end{matrix} \\ \begin{matrix}{\sum }_{j} {s}_{ij} {v}_{j} \leq 0 & & ∀ i ∈ {M}_{r}\end{matrix} \\ \begin{matrix}{\sum }_{j} {s}_{ij} {v}_{j} \geq 0 & & ∀ i ∈ {M}_{p}\end{matrix} \\ {\sum }_{j} {s}_{s , j} {v}_{s} = - 1 \\ {\sum }_{j} {s}_{ATP , j} {v}_{j} \geq {v}_{ATP , min} \\ \begin{matrix}{v}_{j} \geq 0 , & ∀ j ∈ {R}_{irr}\end{matrix}\end{matrix} \qquad ( 2 )$$

where sᵢⱼ is the stoichiometric coefficient of the i^{th} metabolite in the j^{th} reaction, Vⱼ is the flux of the j^{th} reaction, Mᵢ is the set of internal metabolites, Mᵣ is the set of reactants other than the substrate, Mₚ is the set of products, Rᵢᵣᵣ is the set of irreversible reactions. The matrix S={sᵢⱼ} represents the reaction network structure and

$${\sum }_{j} {s}_{ATP , j} {v}_{j} \geq {v}_{ATP , min}$$

satisfies the constraint that a minimum level of ATP is required for maintenance and therefore for the survival of the organism. Equation (2) is linear program and can be solved efficiently by a commercial software program, such as CPLEX, which is an optimization product of ILOG. These commercial software programs run on numerous multiprocessor platforms and can be used to solve linear integer equations, such as those presented herein.

Literature has shown that there are multiple solutions for optimal yield (see Phalakornkule, C., Fry, B., Zhu, T., Kopesel, R., Ataai, M. M. and Domach, M. M. (2000) Biotechnology Progress, 16, 169-175). Such alternative optimal networks can be important to metabolic engineers from the point of view of design. According to one exemplary integer variable set, y={yⱼ}, where yⱼ indicates whether the j^{th} reaction is active or not.

$${y}_{j} = \{ \begin{matrix}1 & if {v}_{j} \neq 0 \\ 0 & otherwise\end{matrix} \qquad ( 3 )$$

According to this exemplary illustration, it is possible to visit the (k+1)^{st} alternate optimum by adding the following constraint successively:

$${\sum }_{j} \vert {y}_{j} - {y}_{j , k}^{*} \vert \geq 1 \qquad ( 4 )$$

where yₖ*={Yⱼₖ*} is k^{th} alternate optimum. The set of successive constraints given by equation (4) ensure that the (k+1)^{st} optimal solution Yₖ₊₁* is different from all the previously visited optima y₁*, y₂*, . . . , yₖ*. This constraint is nonlinear, and therefore the entire optimization problem becomes a mixed-integer-nonlinear program (MINLP). In general, global optimality cannot be guaranteed for a nonlinear optimization problem. Therefore if the constraint (4) can be rewritten as a linear constraint, the problem can be simplified to an MILP, which can then be solved to global optimality.

According to one exemplary illustration, Nk is defined as follows:
Nₖ={j|y_{j,k}*=1} (5)

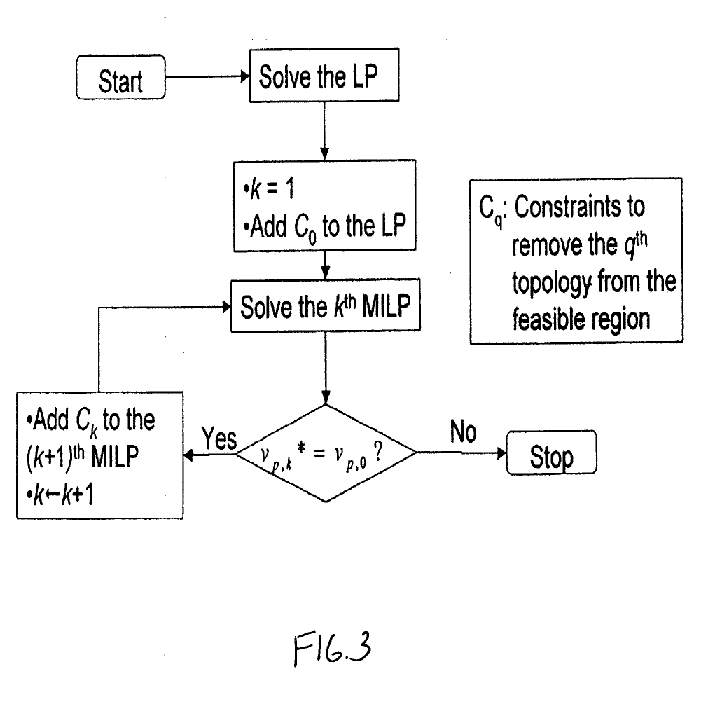

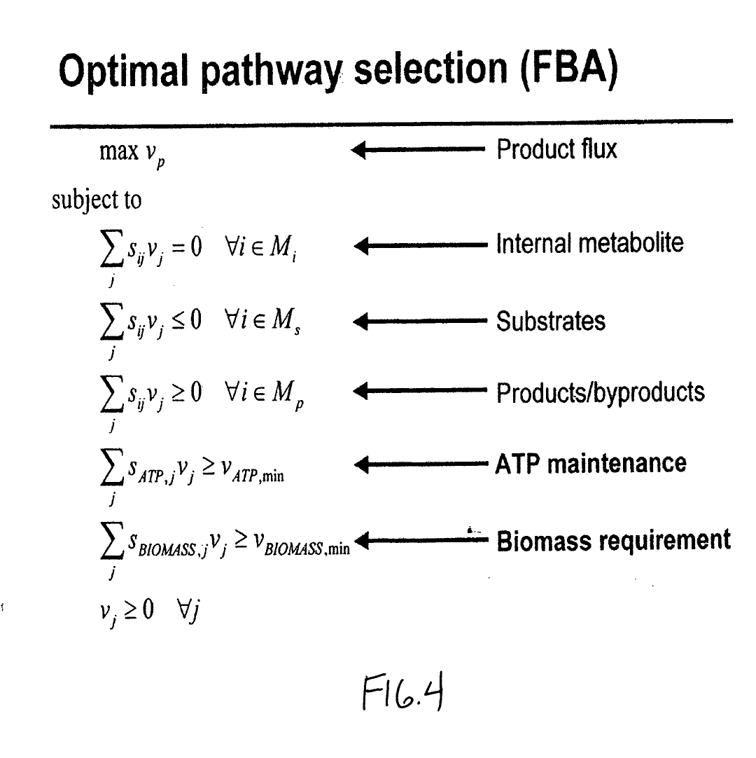

As such, equation (4) can be written as a linear constraint by introducing equation (5) such that:

$${\sum }_{j ∈ {N}_{k}} {y}_{j} - {\sum }_{j ∈ J \backslash {N}_{k}} {y}_{j} \leq \vert {N}_{k} \vert - 1 \qquad ( 6 )$$

wherein |Nₖ| is the cardinality of the set Nₖ. Additional constraints need to be included to ensure that all reactions are irreversible. The rationale for doing so arises from equation (3), where yⱼ=0 if and only if vⱼ=0. As such, if any reaction is actually reversible, it is decomposed into two irreversible reactions, the forward and the reverse. Therefore, the following constraints are added to the linear program:
εvⱼ≦yⱼ≦Evⱼ (7)
yₚ+y_{q} ≦1 (8)
where ε is a small positive number and E is a large number. Reaction p is a reversible reaction, which is decomposed into the corresponding forward and reverse reactions, whose fluxes are yₚ and y_{q} respectively. The constraint given by equation (8) ensures that only one direction of the reversible reaction is active. A flowchart depicting the above process of enumerating multiple topologies with the same theoretical yield is depicted in FIG. 3 and a listing of exemplary optimal pathway formulations according to the FBA process is depicted in FIG. 4.

The iterative procedure to enumerate the multiple optima is described as follows:

Step 1: Solve the linear program (equations (2), (7) and (8)), and get the first optimum, y*ₗ.

Step k: Add constraint (6) to the linear program and resolve it to get y*ₖ until the objective value decreases. Once all the optimal pathways have been obtained, different criteria can be used to discriminate between them and predict the maximal product yield by a stoichiometric analysis. For instance, in certain exemplary embodiments, the ATP production levels of each optimal pathway is considered. A pathway producing more ATP is a favorable choice over other optima because it is energetically more productive. In another exemplary embodiment, the difficulty or effort required to genetically engineer a candidate pathway topology is considered. According to this exemplary embodiment, if the topology is easy to engineer, the engineered strain is made relatively fast, which means this pathway is practically obtainable. Moreover, the number of genes to knockout or add is used as a measure of the genetic engineering ‘cost’ or effort associated with the pathway.

To estimate the effect of cellular maintenance and growth on theoretical yields, two constraints are added into the original formulation.
v_{ATP}≧v_{ATP,min} (9)
v_{biom}≧v_{biom,min} (10)

The maintenance cost, v_{ATP,min}, is formulated in terms of a required ATP flux of 4 mole ATP/mole glucose for E. coli (See Varma, A., and Palsson, B. O. (1993) “Metabolic Capabilities of Escherichia coli: II. Optimal Growth Patterns.” J. Theor. Biol., 165, 503-522). The v_{biom,min}, which is prespecified arbitrarily, denotes the minimum yield of the biomass, which indicates the minimum requirement of the growth.

Because of the underdetermined nature of the FBA problem, more than one network topology could exist for a given maximum yield. In fact, methods to enumerate different topologies (flux distribution maps) with the same yield have been published (See Lee, S., Phalakornkule, C., Domach, M. M., and Grossmann, I. E. (2000) “Recursive MILP model for finding all the alternate optima in LP models for metabolic networks.” Comp. Chem. Eng., 24, 711-716). The problem is formulated as a sequence of MILPs and solved until no new topologies are found. This procedure enumerates all different topologies, but the efficiency is not guaranteed because MILP is an NP-complete problem, and the enumeration is also NP-complete. The worst case of this algorithm is O(2ⁿ), where n is the number of reactions in this problem.

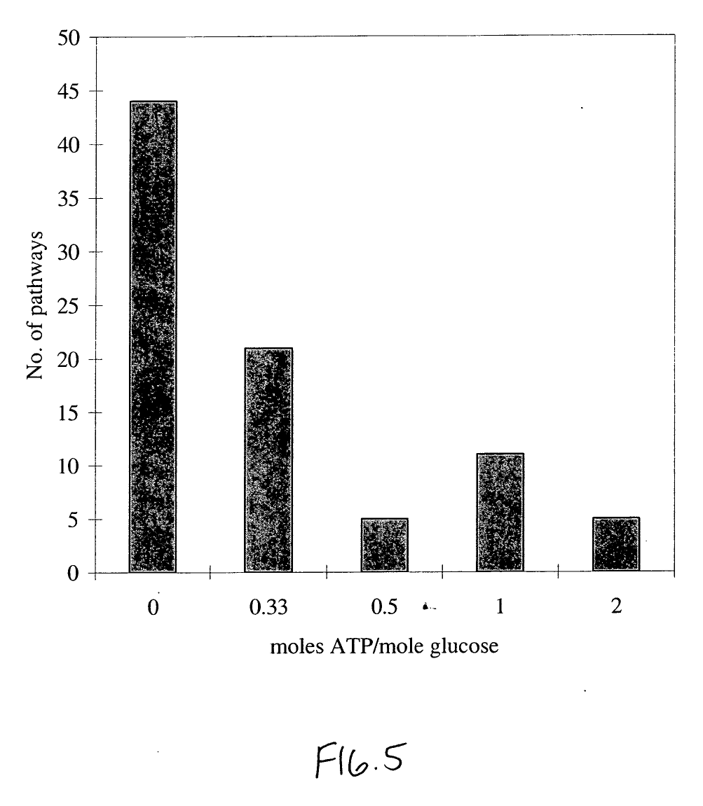

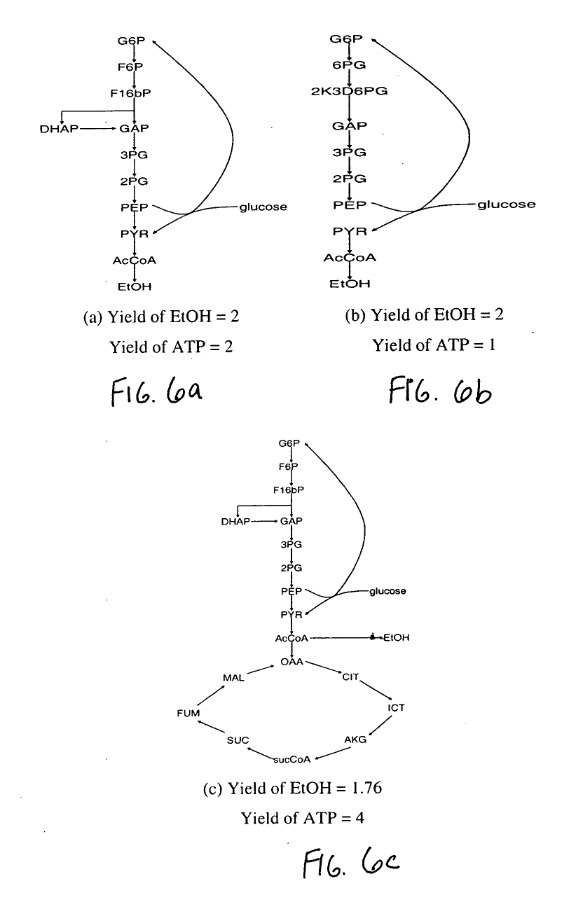

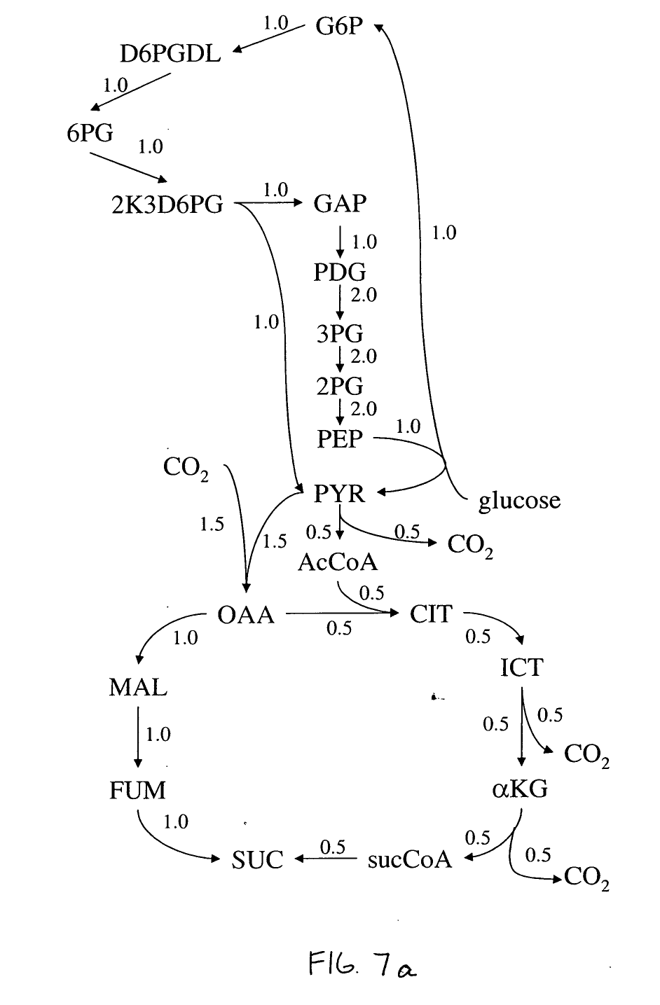

According to one exemplary example, a framework for reverse engineering the metabolic reaction pathway of an E. coli host organism to increase the yield of ethanol production from a glucose based substrate is illustrated. According to this illustration, reactions in the central metabolism of E. coli are considered and the flux of ethanol (given 1 mole of glucose) is maximized. The theoretical yield of ethanol fermentation is 2 moles ethanol/mole glucose without ATP maintenance. 86 different optimal topologies are identified by using the proposed algorithm for maximizing the ethanol flux without ATP maintenance. The ATP production distribution is shown for the different topologies in FIG. 5. The maximum ATP flux among the solutions is 2 moles ATP/mole glucose. The framework identifies five different pathways with this maximum ATP flux, but their topologies are quite similar with the only differences being the use of different cofactors for certain reactions. FIGS. 6(a) and 6(b) show two different topologies for producing ethanol, corresponding to the two well studied glycolysis pathways, EMP (Embden-Meyerhof-Parnas) and ED (Entner-Doudoroff) pathways.

By including a maintenance cost of 4 ATP moles per mole of glucose as reported by Varma and Palsson (see Varma, A. and Palsson, B. O. (1993b) Journal of Theoretical Biology, 165, 503-522), the theoretical yield reduces to 1.76, and only one optimal pathway is obtained, which is shown in FIG. 6(c). This pathway includes the TCA cycle, which is used to generate ATP to satisfy the maintenance constraint.

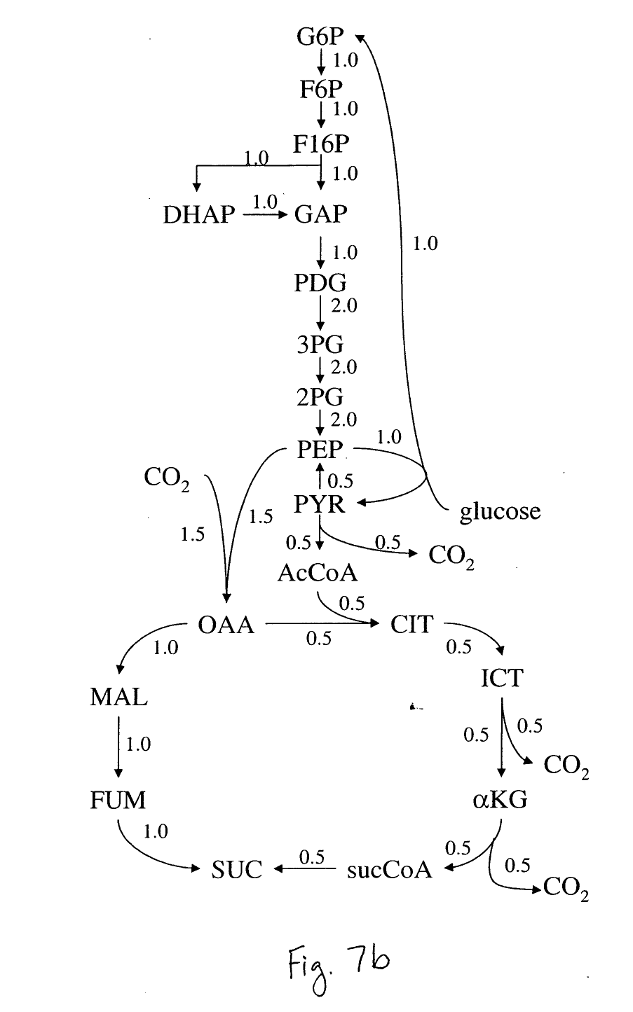

A further exemplary example is depicted with reference to FIGS. 7(a) and 7(b). More particularly, a framework for reverse engineering the metabolic reaction pathway of an E. coli host organism to increase the yield of succinate production from a glucose based substrate is illustrated. According to this exemplary illustration, an optimum yield of 1.5 moles of succinate/1 mol of glucose is produced.

Exemplary embodiments incorporating the present invention construct pathways with maximal yield of a certain product based on the flux balance analysis and multiple solution enumeration technique for MILP. This framework identifies various pathways for producing the product (e.g., ethanol in the above illustration), and includes the simplest linear pathway for producing the product. The ATP maintenance constraint is then added to estimate the real maximum yield and typically reduces the yield of the product because of the carbon lost during the process to produce ATP. This framework can be applied on metabolic pathway design, and by calculating the gene knockouts for all the different topologies, it is possible to find the most economical one that has the ability to secrete the desired product. It is also possible to add genes from other organisms into the E. coli genome, and predict the yield of the engineered strain.

According to exemplary embodiments, several factors are considered when comparing optimal metabolic pathways of single organisms within the metabolic network database. For instance, when selecting an optimal metabolic pathway of an organism, the number of genes that must be added and/or deleted should be minimized. Moreover, the ATP maintenance cost must be considered, as well as the organism's tolerance to high concentrations of substrate/product. Available recombinant DNA techniques should also be considered. Furthermore, while the above exemplary illustration demonstrates E. coli as the host organism, it is envisioned that those skilled in the art may utilize several known prokaryotic or eukaryotic microbes as the host organism without straying from the scope of the present invention. Moreover, in further exemplary embodiments, the host organism may be a heterotrophic organism and the enzymes added to modify its reaction pathway may be from an autotrophic source.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of enhancing product yield, comprising:
selecting a host organism adapted to produce the product as a function of metabolism in the presence of a substrate;
determining a plurality of optimal reaction pathways by searching a database and executing an optimization algorithm, the optimal reaction pathways taking place in the host organism to produce the product from the substrate; and
ranking and enumerating the optimal reaction pathways.

2. The method of claim 1, wherein the optimization algorithm comprises modifying genes of the host organism.

3. The method of claim 2, wherein the modifying comprises adding or subtracting an enzyme.

4. The method of claim 3, wherein the added enzymes are obtained from an autotroph and the host organism is a heterotroph.

5. The method of claim 1, wherein the ranking comprises one or more of, evaluating kinetics, evaluating ATP production, and minimizing changes to the host organism.

6. The method of claim 1, wherein the optimization algorithm comprises applying a flux balance analysis.

7. The method of claim 6, wherein the flux balance analysis comprises conducting a mixed-integer program calculation.

8. The method of claim 6, wherein the flux balance analysis comprises maximizing yield of the product.

9. The method of claim 7, wherein the mixed-integer program comprises a linear calculation.

10. The method of claim 1, wherein the host organism comprises a prokaryotic microbe.

11. The method of claim 10, wherein the prokaryotic microbe comprises Escherichia coli.

12. The method of claim 1, wherein the product comprises ethanol.

13. The method of claim 1, wherein the substrate comprises a sugar.

14. A method of ranking a plurality of optimal reaction pathways, comprising:
accessing a database containing a plurality of metabolic reactions;
conducting a flux balance analysis comprising solving a mixed-integer program calculation to determine the plurality of optimal reaction pathways from the plurality of metabolic reactions; and
ranking and enumerating the optimal reaction pathways by one or more of, evaluating kinetics, evaluating ATP production, and minimizing changes to the host organism.

15. The method of claim 14, further comprising modifying genes of the host organism by adding one or more of the metabolic reactions to the host organism.

16. The method of claim 15, wherein the modifying comprises adding or subtracting an enzyme.

17. The method of claim 16, wherein the added enzymes are obtained from an autotroph and the host organism is a heterotroph

18. The method of claim 14, wherein the flux balance analysis comprises maximizing yield of the product.

19. The method of claim 14, wherein the host organism comprises a prokaryotic microbe.

20. The method of claim 19, wherein the prokaryotic microbe comprises Escherichia coli.

21. The method of claim 14, wherein the product comprises ethanol.

22. The method of claim 14, wherein the substrate comprises a sugar.
